(19) United States
(12) Patent Application Publication
(10) Pub. No.: US 2018/0143206 A1
(43) Pub. Date: May 24, 2018

(54) CXCL10 AS PREDICTIVE BIOMARKER OF RENAL TRANSPLANT ACUTE REJECTION AND DIAGNOSTIC BIOMARKER OF ANTIBODY-MEDIATED REJECTION (ABMR)

(71) Applicants: INSERM (INSTITUT NATIONAL DE LA SANTÉ ET DE LA RECHERCHE MÉDICALE), Paris (FR); UNIVERSITÉ PARIS DESCARTES, Paris (FR); ASSISTANCE PUBLIQUE-HÔPITAUX DE PARIS (APHP), Paris (FR); CENTRE NATIONAL DE LA RECHERCHE SCIENTIFIQUE (CNRS, Paris (FR)

(72) Inventors: Dany ANGLICHEAU, Paris (FR); Fabiola TERZI, Paris (FR); Christophe LEGENDRE, Paris (FR)

(21) Appl. No.: 15/571,545

(22) PCT Filed: May 4, 2016
(86) PCT No.: PCT/EP2016/060022

§ 371 Date: Nov. 3, 2017

(30) Foreign Application Priority Data
May 5, 2015 (EP) 15305688.2

(51) Int. Cl. G01N 33/68; A61K 39/395; G01N 33/50

(52) U.S. Cl. CPC G01N 33/6893; A61K 39/3955; G01N 33/5091; A61K 2300/00; G01N 2800/245; G01N 2800/50; G01N 2800/52; G01N 2333/522

(57) ABSTRACT

The invention relates to a method for determining whether a renal transplanted patient is at risk of acute rejection, comprising a step of determining the expression level of the CXCL10 polypeptide in a urine sample obtained from said patient until 3-months post-transplantation. The invention also relates to a method for diagnosing antibody-mediated rejection (ABMR) in a renal transplanted patient, comprising a step of determining the expression level of the CXCL10 polypeptide in a urine sample obtained from said patient.

### FIELD OF THE INVENTION

The invention relates to the field of medicine, and more particularly to the early prediction of acute rejection in a renal transplanted patient as well as the diagnosis of ABMR.

### BACKGROUND OF THE INVENTION

More than sixty years after the first kidney transplant was performed, routine screening after kidney transplantation still relies on monitoring renal allograft function through serum creatinine and urine protein measurements.¹ Based on the rationale that detecting allograft dysfunction as soon as possible will allow timely diagnosis and treatment and therefore improve outcomes, studies in the last decade have focused on developing noninvasive tests that rely on easily accessible biological fluids, such as urine and blood, and that could ultimately be used for noninvasive serial monitoring.²

Urinary chemokines, mainly the CXCR3-binding chemokines known as chemokine (C—X—C motif) ligand 9 (CXCL9) and CXCL10, are secreted by infiltrating inflammatory cells, renal tubular and endothelial cells³ and have been thoroughly investigated as noninvasive biomarkers of acute rejection (AR).⁴⁻¹³ Many observational cross-sectional studies performed at the time of indication biopsies have clearly demonstrated their diagnostic performance in the setting of acute T cell-mediated rejection (TCMR) of renal allografts,^{5, 9, 10, 12, 15}. Using a cross-sectional design, Schaub et al first reported the association of CXCL9 and CXCL10 with subclinical tubulitis,¹⁰ a result that was subsequently confirmed by independent studies¹³ and supported the hypothesis that serial monitoring of urine chemokine levels, even in clinically stable kidney transplant recipients, may provide additional information for assessing allograft status.

Unfortunately, very few studies have evaluated the performance of these biomarkers in predicting AR during longitudinal follow-up with sequential urine specimens obtained at precise time points.^{16, 8}

Moreover, in renal transplantation, the antibody-mediated rejection (ABMR) is currently a major thread for the graft survival. The medical community stresses the necessity of developing new biomarkers in diagnosing ABMR because the current criteria are neither necessary nor sufficient: the presence of donor specific antibodies (DSA) in the plasma of patients is a pre-requisite for the diagnosis of ABMR, but it is not always associated with ABMR. It results that it is likely that many patients with ABMR are underdiagnosed and not appropriately treated. Yet, early and accurate diagnosis of ABMR is thus important because a specific treatment, different from that of TCMR, can be proposed for these patients including anti-CD20 antibodies (rituximab). Otherwise, in the absence of appropriate treatment, the patients can lose their graft rapidly. In addition, when the diagnostic can be made with certainty, undue over-immunosuppression will be prevented.

### SUMMARY OF THE INVENTION

In a first aspect, the invention relates to a method for determining whether a renal transplanted patient is at risk of acute rejection, comprising the steps of (i) determining the expression level of the CXCL10 polypeptide in a urine sample obtained from said patient until 3-months post-transplantation, and (ii) comparing said expression level with a predetermined reference value, wherein the expression level of the CXCL10 polypeptide determined at step (i) is lower the predetermined reference value is indicative for said patient of not being at risk of acute rejection or of having a decreased risk of acute rejection.

In a second aspect, the invention relates to a method for diagnosing antibody-mediated rejection (ABMR) in a renal transplanted patient, comprising the steps of (i) determining the expression level of the CXCL10 polypeptide in a urine sample obtained from said patient, and (ii) comparing said expression level with a predetermined reference value, wherein the expression level of the CXCL10 polypeptide determined at step (i) is lower the predetermined reference value is indicative for said patient of not having ABMR.

In a third aspect, the invention relates to a method for adjusting the immunosuppressive treatment administered to a renal transplanted patient following its transplantation, comprising the steps of: (i) performing the method for determining whether a renal transplanted patient is at risk of acute rejection of the invention or the method for diagnosing ABMR of the invention, and (ii) adjusting the immunosuppressive treatment.

In a fourth aspect, the invention relates to a method for preventing ABMR or progression of ABMR in a renal transplanted patient, comprising the steps of: (i) performing the method for diagnosing ABMR of the invention, and (ii) administering to said patient a therapeutically effective amount of a compound selected from the group consisting of anti-thymocyte globulin, monoclonal anti-CD20 antibodies, proteasome inhibitor, anti-C5 antibodies and plasmapheresis.

### DETAILED DESCRIPTION OF THE INVENTION

The invention addresses these needs, as it relates to methods and treatment approaches useful in the prediction and prevention of development and progression of acute rejection in renal transplanted patients (e.g. ABMR) as well as in the diagnosis of ABMR.

The inventors have demonstrated that the expression level of CXCL10 polypeptide in a urine sample is useful to predict acute rejection, until one half year before said acute rejection. The inventors have also shown that expression level of CXCL10 polypeptide in a urine sample is also useful for diagnosing renal transplanted patients with ABMR.

Firstly, the inventors investigated CXCL10 levels in 1,719 urine samples, from 300 consecutive kidney recipients, collected during the first post-transplant year and assessed their predictive value for subsequent AR using 773 biopsies (479 protocol and 294 for cause).

The trajectories of urinary CXCL10 during the first 400 days post-transplantation showed an early increase in patients who subsequently developed AR. Early urinary CXCL10 levels were strongly increased in patients with subsequent AR (p=0.0005 and p=0.0009 at 1 month and 3 months post-transplantation, respectively). Using time-dependent receiver operating characteristic analyses, highly AR-predictive CXCL10 cutoff levels were defined; 400 days post-transplantation, the AR-free allograft survival rates were 90% and 54% in patients with urinary CXCL10:creatinine (CXCL10:Cr) levels <2.79 ng/mmoL and >2.79 ng/mmoL at 1 month, respectively (P<0.0001), and 88% and 56% in patients with urinary CXCL10:Cr levels <5.32 ng/mmoL and >5.32 ng/mmoL at 3 months post-transplantation (P<0.0001), respectively. In 220 clinically and histologically stable patients, multivariate Cox proportional hazard analysis, adjusted for donor specific antibody status and history of delayed graft function, confirmed that CXCL10:Cr at 3 months post-transplantation predicted AR, independent of concomitant protocol biopsy results (p=0.009). Urinary CXCL10:Cr levels at 1 and 3 months strongly predict the risk of clinical and subclinical AR in the first post-transplant year, even in clinically and histologically stable patients.

Secondly, the inventors investigated urinary CXCL9 and CXCL10 levels in a highly sensitized cohort of 244 renal allograft recipients (67 with pre-formed donor-specific antibody [DSAs]) with 281 indication biopsies. They assessed the benefit of adding these biomarkers to conventional models for diagnosing/prognosticating ABMR.

Urinary CXCL9 and CXCL10 levels, normalized (CXCL9: Cr and CXCL10: Cr ratios) or not to urine creatinine levels, correlated well with the extent of tubulointerstitial (i+t score, all P<0.0001) and microvascular (g+ptc score, all P<0.0001) inflammation. In addition to be diagnostic of TCMR ([area under the curve {AUC}]=0.80; 95% confidence interval [CI]: 0.68-0.92; P=7.1E-04), CXCL10: Cr also noninvasively diagnosed ABMR with high accuracy (AUC=0.76; 95% CI: 0.69-0.82; P=1.5E-10) even in the total absence of tubulointerstitial inflammation (AUC=0.70; 95% CI: 0.61-0.79; P=4.8E-5). Although the mean fluorescence intensity of the immunodominant DSA (iDSA) noninvasively diagnosed ABMR (AUC=0.75; 95% CI: 0.68-0.82; P=5.6E-12), combining the urinary CXCL10: Cr ratio with iDSA levels improved the noninvasive diagnosis of ABMR (AUC=0.83; P=2.5E-15). At time of ABMR, urinary CXCL10: Cr ratio was independently associated with an increased risk of graft loss.

Urinary CXCL10: Cr ratio is not only associated with tubulointerstitial but also with microvascular inflammation of the renal allograft. The combination of the urinary CXCL10: Cr ratio with DSA monitoring significantly improves the noninvasive diagnosis of ABMR and helps stratification of patients at high risk for graft loss.

### Definitions

Throughout the specification, several terms are employed and are defined in the following paragraphs.

As used herein, the term “determining” includes qualitative and/or quantitative detection (i.e. detecting and/or measuring the expression level) with or without reference to a control or a predetermined value. As used herein, “detecting” means determining if the CXCL10 polypeptide is present or not in a biological sample and “measuring” means determining the amount of CXCL10 polypeptide in a biological sample. Typically the expression level may be determined for example by immunoassays such as an ELISA performed on a biological sample, such as a urine sample obtained from the patient.

As used herein, the term “CXCL10” refers to the chemokine (C—X—C motif) ligand 10 also known as Interferon gamma-induced protein 10 (IP-10) which is a small cytokine belonging to the CXC chemokine family. The naturally occurring human CXCL10 polypeptide has an aminoacid sequence of 98 amino acids provided in the NCBI database under accession number NP 001556 and is shown as follows (SEQ ID NO: 1):

### MNQTAILICC LIFLTLSGIQ GVPLSRTVRC TCISISNQPV NPRSLEKLEI IPASQFCPRV EIIATMKKKG EKRCLNPESK AIKNLLKAVS KERSKRSP

### Prognostic Methods of the Invention

In a first aspect, the invention relates to a method for determining whether a renal transplanted patient is at risk of acute rejection, comprising a step of determining the expression level of the CXCL10 polypeptide in a urine sample obtained from said patient until 3-months post-transplantation.

As used herein, the term “risk” refers to the probability that an event will occur over a specific time period, such as the onset of transplant rejection, and can mean a subject's “absolute” risk or “relative” risk. Absolute risk can be measured with reference to either actual observation post-measurement for the relevant time cohort, or with reference to index values developed from statistically valid historical cohorts that have been followed for the relevant time period. Relative risk refers to the ratio of absolute risks of a patient compared either to the absolute risks of low risk cohorts or an average population risk, which can vary by how clinical risk factors are assessed. Odds ratios, the proportion of positive events to negative events for a given test result, are also commonly used (odds are according to the formula p/(1−p) where p is the probability of event and (1−p) is the probability of no event). “Risk determination” in the context of the invention encompasses making a prediction of the probability, odds, or likelihood that an event may occur. Risk determination can also comprise prediction of future clinical parameters, traditional laboratory risk factor values, such age, sex mismatch, HLA-testing, etc . . . ; either in absolute or relative terms in reference to a previously measured population. The methods of the invention may be used to make categorical measurements of the risk of transplant rejection, thus defining the risk spectrum of a category of transplanted patient defined as being at risk of transplant rejection.

As used herein, the term “acute rejection” is the rejection by the immune system of a tissue transplant recipient when the transplanted tissue is immunologically foreign. Acute rejection is characterized by infiltration of the transplant tissue by immune cells of the recipient, which carry out their effector function and destroy the transplant tissue. The onset of acute rejection is rapid and generally occurs in humans within a few weeks after transplant surgery. Generally, acute rejection can be inhibited or suppressed with immunosuppressive drugs such as rapamycin, everolimus, cyclosporin, tacrolimus, mycophenolic acid, anti-CD25 monoclonal antibody and the like.

The term “transplantation” and variations thereof refers to the insertion of a transplant (also called graft) into a recipient, whether the transplantation is syngeneic (where the donor and recipient are genetically identical), allogeneic (where the donor and recipient are of different genetic origins but of the same species), or xenogeneic (where the donor and recipient are from different species). Thus, in a typical scenario, the host is human and the graft is an allograft, derived from a human of different genetic origins. In another scenario, the graft is derived from a species different from that into which it is transplanted, including animals from phylogenically widely separated species.

In one embodiment, the urine sample is obtained 10-days post-transplantation, 1-month post-transplantation, or 3-months post-transplantation.

In one embodiment, said method comprises the followings steps of:
(i) determining the expression level of the CXCL10 polypeptide in a urine sample obtained from said patient, and
(ii) comparing said expression level with a predetermined reference value,
wherein the expression level of the CXCL10 polypeptide determined at step (i) is lower the predetermined reference value is indicative for said patient of not being at risk of acute rejection or of having a decreased risk of acute rejection.

As used herein, the term “predetermined reference value” refers to the expression level of the CXCL10 polypeptide in urine samples obtained from the general population or from a selected population of subjects. The predetermined reference value can be a threshold value or a range. For example, the selected population may be comprised of apparently healthy transplanted patient, such as individuals who have not previously had any sign or symptoms indicating the outcome of a renal graft rejection, more particularly acute rejection.

A “predetermined reference level” may be determined, for example, by determining the expression level of CXCL10 polypeptide, in a corresponding urine sample obtained from one or more control subject(s) (e.g., not suffering from graft rejection or known not to be susceptible to such a disease). When such a predetermined reference level is used, a lower or decreased levels determined in a urine sample (i.e. a test sample obtained from the patient) is indicative for example that said patient is not at risk of having acute rejection. The predetermined reference level may be established based upon comparative measurements between apparently healthy patients (e.g. patients classified with normal biopsy) and patients with established graft rejection (including acute T-cell mediated rejection (a-TCMR) or acute antibody-mediated rejection (a-ABMR)).

As used herein, a “lower” or “decreased” level refers to an expression level in a biological sample (i.e. urine sample obtained from the patient) which is below the predetermined reference level (e.g., CXCL10 concentration that discriminates patients at high or low risk of having an acute rejection as above-defined).

As used herein, the term “urine sample” refers to a biological sample obtained for the purpose of in vitro evaluation. A urine sample can be optionally pre-treated or processed prior to be used.

In one embodiment, the expression level of the CXCL10 polypeptide is expressed as the ratio of expression levels of CXCL10 polypeptide to urinary creatinine (CXCL10:Cr).

In a second aspect, the invention relates to a method for diagnosing antibody-mediated rejection (ABMR) in a renal transplanted patient, comprising a step of determining the expression level of the CXCL10 polypeptide in a urine sample obtained from said patient.

In one embodiment, said method comprises the followings steps of:
(i) determining the expression level of the CXCL10 polypeptide in a urine sample obtained from said patient, and
(ii) comparing said expression level with a predetermined reference value,
wherein the expression level of the CXCL10 polypeptide determined at step (i) is lower the predetermined reference value is indicative for said patient of not having ABMR.

In one embodiment, the expression level of the CXCL10 polypeptide is expressed as the ratio of expression levels of CXCL10 polypeptide to urinary creatinine (CXCL10:Cr).

In another aspect, the invention relates to the use of urinary CXCL10 polypeptide as a biomarker of ABMR.

The term “biomarker”, as used herein, refers generally to a molecule, i.e., a protein, the expression of which in a biological sample from a patient can be detected by standard methods in the art (as well as those disclosed herein), and is predictive or denotes a condition of the patient from which it was obtained.

In one embodiment, the invention relates to the use of urinary CXCL10 polypeptide as a biomarker of ABMR in a transplanted renal patient.

### Methods for Determining the Expression Level of the Biomarker of the Invention

Determination of the expression level of CXCL10 polypeptide may be performed by a variety of techniques. Generally, the expression level as determined is a relative expression level. For example, the determination of the expression level of CXCL10 polypeptide may comprise a step of contacting the biological sample with selective binding reagents such as antibodies, and thereby detecting the presence, or measuring the amount, of polypeptide of interest originally in said biological sample. Contacting may be performed in any suitable device, such as a plate, microtiter dish, test tube, well, glass, column, and so forth.

In one embodiment, the contacting is performed on a substrate coated with the reagent. The substrate may be a solid or semi-solid substrate such as any suitable support comprising glass, plastic, nylon, paper, metal, polymers and the like. The substrate may be of various forms and sizes, such as a slide, a membrane, a bead, a column, a gel, etc. The contacting may be made under any condition suitable for a detectable complex, such as an antibody-antigen complex, to be formed between the reagent and the polypeptides of the biological sample.

The presence of the CXCL10 may be detected using standard electrophoretic and immunodiagnostic techniques, including immunoassays such as competition, direct reaction, or sandwich type assays. Such assays include, but are not limited to, Western blots; agglutination tests; enzyme-labelled and mediated immunoassays, such as ELISAs; biotin/avidin type assays; radioimmunoassays; immunoelectrophoresis; immunoprecipitation, etc. The reactions generally include revealing labels such as fluorescent, chemiluminescent, radioactive, enzymatic labels or dye molecules, or other methods for detecting the formation of a complex between the antigen and the antibody or antibodies reacted therewith. Labels are known in the art that generally provide (either directly or indirectly) a signal.

More particularly, an ELISA method may be used, wherein the wells of a microtiter plate are coated with an antibody against the protein to be tested. A biological sample containing or suspected of containing the biomarker is then added to the coated wells. After a period of incubation sufficient to allow the formation of antibody-antigen complexes, the plate(s) can be washed to remove unbound moieties and a detectably labelled secondary binding molecule added. The secondary binding molecule is allowed to react with any captured sample marker protein, the plate washed and the presence of the secondary binding molecule detected using methods well known in the art.

Measuring the expression level of a biomarker protein such as CXCL10 (with or without immunoassay-based methods) may also include separation of the proteins: centrifugation based on the protein's molecular weight; electrophoresis based on mass and charge; HPLC based on hydrophobicity; size exclusion chromatography based on size; and solid-phase affinity based on the protein's affinity for the particular solid-phase that is use. Once separated, CXCL10 may be identified based on the known “separation profile” e.g., retention time, for that protein and measured using standard techniques.

In one embodiment, the selective binding reagent is generally an antibody that may be polyclonal or monoclonal, preferably monoclonal. Monoclonal antibodies directed against CXCL10 are also well known such as the monoclonal antibody MAB266-SP human CXCL10/IP-10 MAb (Clone 33036) commercialized by R&D Systems.

Additionally, CXCL10 ELISA Kits are also well known such as IP10 Quantikine ELISA, #DIP100 also commercialized by R&D Systems.

As used herein, the term “monoclonal antibody” refers to a population of antibody molecules that contains only one species of antibody combining site capable of immunoreacting with a particular epitope. A monoclonal antibody thus typically displays a single binding affinity for any epitope with which it immunoreacts. A monoclonal antibody may therefore contain an antibody molecule having a plurality of antibody combining sites, each immunospecific for a different epitope, e.g. a bispecific monoclonal antibody. Although historically a monoclonal antibody was produced by immortalization of a clonally pure immunoglobulin secreting cell line, a monoclonally pure population of antibody molecules can also be prepared by the methods of the invention.

Laboratory methods for preparing monoclonal antibodies are well known in the art (see, for example, Harlow et al., 1988). Monoclonal antibodies (mAbs) may be prepared by immunizing purified Notch3 into a mammal, e.g. a mouse, rat, human and the like mammals. The antibody-producing cells in the immunized mammal are isolated and fused with myeloma or heteromyeloma cells to produce hybrid cells (hybridoma). The hybridoma cells producing the monoclonal antibodies are utilized as a source of the desired monoclonal antibody. This standard method of hybridoma culture is described in Kohler and Milstein (1975).

In one embodiment, the methods further comprises a step of determining another biomarker useful for diagnosing ABMR such as donor-specific antibodies (DSA). Usually, said DSA may be detected in a blood sample obtained from the renal transplanted patient.

Accordingly, the invention also relates to a kit for performing a method above-mentioned, wherein said kit comprises (i) means for determining the expression level of the CXCL10 in a urine sample obtained from said renal transplanted patient, and (ii) means for determining the expression level of donor-specific antibodies (DSA).

Typically, means for determining the expression level of the CXCL10 are CXCL10 ELISAs. Typically, means for determining the expression level of donor-specific antibodies (DSA) are single antigen-flow beads assays as described in the Section EXAMPLES.

### Methods for Adjusting an Immunosuppressive Treatment

The invention further provides methods for developing personalized treatment plans. Information gained by way of the methods described above can be used to develop a personalized treatment plan for a transplant recipient.

Accordingly, in a further aspect, the invention relates to a method for adjusting the immunosuppressive treatment administered to a renal transplanted patient following its transplantation, comprising the steps of: (i) performing the method for determining whether a renal transplanted patient is at risk of acute rejection or the method for diagnosing ABMR of the invention, and (ii) adjusting the immunosuppressive treatment.

The methods can be carried out by, for example, using any of the methods for determining risk described above and, in consideration of the results obtained, designing a treatment plan for the transplant recipient.

If CXCL10 is increased in a urine sample obtained from a patient of interest, this indicates that said patient is at risk for an undesirable clinical outcome in the following year (e.g., acute rejection) and/or said patient is suffering from ABMR. Therefore, said patient is a candidate for treatment with an effective amount of an immunosuppressive treatment (e.g. by an anti-rejection agent). On the contrary, a low CXCL10 level is indicative of a reduced risk of transplant rejection. Moreover, depending on the expression level CXCL10 (i.e. low level or high level of CXCL10 in the analyzed urine sample), the patient may require a treatment regime that is more or less aggressive than a standard regimen, or it may be determined that the patient is best suited for a standard regimen. For instance, a patient with low levels of CXCL10 in a urine sample may avoid an immunosuppressive treatment (or require a less aggressive regimen) and their associated side effects.

Reducing the level and the production of the DSA and/or protecting the allograft may be achieved using any suitable medical means known to those skilled in the art.

In one embodiment, such reduction and protection comprise a therapeutic intervention with the patient such as increase in the maintenance immunosuppressive regimen, administration of anti-thymocyte globulin (ATG), monoclonal anti-CD20 antibodies (rituximab), proteasome inhibitor (bortezomib), anti-C5 antibodies (eculizumab), intravenous administration of immunoglobulins and plasmapheresis.

### Therapeutic Methods and Uses

In a further aspect, the invention relates to a method for preventing ABMR or progression of ABMR in a renal transplanted patient, comprising the steps of: (i) performing the method for diagnosing ABMR of the invention, and (ii) administering to said patient a therapeutically effective amount of a compound selected from the group consisting of anti-thymocyte globulin (ATG), monoclonal anti-CD20 antibodies (rituximab), proteasome inhibitor (bortezomib), anti-05 antibodies (eculizumab), and plasmapheresis.

By “therapeutically effective amount” is meant an amount sufficient to achieve a concentration of compound which is capable of preventing or slowing down the disease to be treated. Such concentrations can be routinely determined by those of skilled in the art. The amount of the therapeutic agent actually administered will typically be determined by a physician or a veterinarian, in the light of the relevant circumstances, including the condition to be treated, the chosen route of administration, the actual compound administered, the age, weight, and response of the patient, the severity of the subject's symptoms, and the like. It will also be appreciated by those of skilled in the art that the dosage may be dependent on the stability of the administered compound.

In one embodiment, said compound is selected from the group consisting of anti-thymocyte globulin (ATG), monoclonal anti-CD20 antibodies (rituximab), proteasome inhibitor (bortezomib), anti-C5 antibodies (eculizumab), and plasmapheresis.

The compounds of the invention may be administered by any means that achieve the intended purpose. For example, administration may be achieved by a number of different routes including, but not limited to subcutaneous, intravenous, intradermal, intramuscular, intraperitoneal, or subcutaneous use. Parenteral route is particularly preferred.

Dosages to be administered depend on individual needs, on the desired effect and the chosen route of administration. It is understood that the dosage administered will be dependent upon the age, sex, health, and weight of the recipient, concurrent treatment, if any, frequency of treatment, and the nature of the effect desired. The total dose required for each treatment may be administered by multiple doses or in a single dose.

The doses used for the administration can be adapted as a function of various parameters, and in particular as a function of the mode of administration used, of the relevant pathology, or alternatively of the desired duration of treatment. For example, it is well within the skill of the art to start doses of the compounds at levels lower than those required to achieve the desired therapeutic effect and to gradually increase the dosage until the desired effect is achieved. However, the daily dosage of the therapeutic agent may be varied over a wide range from 0.01 to 1,000 mg per adult per day. Preferably, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the subject to be treated.

The invention will be further illustrated by the following figures and examples. However, these examples and figures should not be interpreted in any way as limiting the scope of the present invention.

### FIGURES

FIG. 1: Urinary biomarkers are diagnostic of both TCMR and ABMR. Box-and-whisker plots show the log (natural)-transformed urinary biomarker levels in 203 matched urine/biopsy samples from patients with allograft dysfunction but no rejection (DNR), 10 matched urine/biopsy samples from patients with T cell-mediated rejection (TCMR), 37 matched urine/biopsy samples from patients with pure ABMR and 31 matched urine/biopsy samples from patients with mixed rejection (mixed). P values are based on the Kruskal-Wallis test. Stars depict pairwise group comparisons by means of Dunn's post-test (** P<0.01; *** P<0.001).

FIG. 2: Acute rejection-free allograft survival according to early CXCL10 levels. Kaplan-Meier estimates of acute rejection-free allograft survival, stratified according to selected levels of urinary CCXCL10:Cr are shown at month-1 (A) and at month-3 (B).

For each curve, only patients with no previous acute rejection at the time of urine sampling were included.

### EXAMPLE 1: URINARY CXCL10 INDEPENDENTLY IMPROVES THE NONINVASIVE DIAGNOSIS OF ANTIBODY-MEDIATED KIDNEY ALLOGRAFT REJECTION

Material & Methods

Study population: Beginning in February 2011, midstream urine samples were obtained immediately before clinically indicated renal allograft biopsies. This single center study was approved by the ethics committee of Ile-de-France XI (#13016), and all participating patients provided written informed consent. All patients with indication biopsy and a corresponding urine specimen were prospectively included from February 2011 to February 2013. Patients with inconclusive biopsies were excluded.

Sixty-seven patients had DSAs at time of transplantation and had been included in our high-risk transplant program. All these high-risk patients received an induction therapy, either by rabbit ATG (Thymoglobuline®, Sanofi, France, [n=56]), or Basiliximab (Simulect®, Novartis Pharma AG, Basel, Switzerland, [n=11]) and four courses of IVIg in addition to a conventional triple-drug immunosuppressive regimen consisting of calcineurin inhibitors, mycophenolic acid and prednisone. From 2006 onward, patients with DSA at day 0 received additional prophylactic rituximab therapy (Mabthera®, Roche pharmaceuticals, Basel, Switzerland), together with plasmapheresis.

Most of the remaining patients received a conventional triple-drug calcineurin-inhibitor-based immunosuppressive regimen.

Acute TCMRs were treated with high-dose steroids. Patients with acute ABMR received rituximab (325 mg/m²), high-dose steroids, and underwent plasma exchanges followed by four courses of IVIg.

DSAs were present at time of biopsy in 41% (110/268) of cases. 30% ( 33/110) of iDSAs were anti-class I DSAs and 70% ( 77/110) were anti-class II DSAs. Mean (±SEM) MFI of the iDSA was 5074±543 at time of biopsy (2645±394 for anti-class I DSAs and 6172±734 for anti class II DSAs).

Urine Sample Collection:

Urine specimens were collected immediately before the clinically indicated biopsy and centrifuged at 1,000 g for 10 minutes within 4 hours of collection. The supernatant was collected after centrifugation and stored with protease inhibitors at −80° C.

Urine Protein Analyses:

Frozen aliquots of urine supernatants were used without any dilution and tested by ELISA for CXCL10 (IP10 Quantikine ELISA, #DIP100, R&D Systems), according to the manufacturer's instructions, and for CXCL9 (Human CXCL9/MIG DuoSet, R&D Systems), as recommended by the CTOT-01 study. The mean minimum detectable level in the ELISA assay was 1.67 pg/mL for CXCL10, and urine samples with a chemokine concentration below this value were included in the analysis as half the detection limit. For CXCL9, the urine samples with chemokine levels below the detection limit were included as half the minimum value detected (7 pg/mL).

Measurement of urine creatinine was performed in the same sample using a Hitachi 917 analyzer (Roche Diagnostics). The results were normalized or not to the urinary creatinine level, and, consequently, 4 biomarkers were analyzed: CXCL10 (expressed in pg/mL), CXCL10: creatinine ratio (CXCL10: Cr, expressed in ng protein/mmoL urine creatinine), CXCL9 (pg/mL), and CXCL9: creatinine ratio (CXCL9: Cr, ng protein/mmoL).

Urinalysis was systematically performed at the time of urine collection, and leukocyturia was recorded and classified into 4 categories (<10⁴, 10⁵, 10⁶, >10⁶ leucocytes/mL).

Renal Allograft Biopsy Histology:

Clinically indicated biopsy specimens were fixed in formalin, acetic acid, and alcohol (FAA) and embedded in paraffin. Tissue sections were stained with hematoxylin and eosin, Masson trichrome, periodic acid Schiff reagent, and Jones for light microscopy evaluation. C4d immunohistochemical staining was systematically performed (rabbit anti-human monoclonal anti-C4d, Clinisciences, 1/200 dilution).

Renal allograft biopsies were classified using the Banff 2007 update of the Banff 1997 classification. For the purpose of this study, biopsies were categorized into one of four groups according to the histological diagnosis: pure ABMR, mixed rejection, TCMR, or DNR. Pure ABMR were defined as ABMR with no tubulointerstitial inflammation (i+t score=0). Mixed rejections include all ABMR with i+t score≠0, including primarily ABMR with minimal tubulointerstitial inflammation (n=21) and ABMR with TCMR≥1a (n=10). In several analyses, mixed rejections were included in the ABMR category because they required the same clinical management and therapeutic interventions as ABMR.

Donor-Specific Antibodies:

The presence of circulating DSAs at the time of biopsy was analyzed using single-antigen flow bead assays (One Lambda, Canoga Park, Calif.) on the Luminex platform as previously described. Beads showing a normalized MFI greater than 500 were considered positive. For each patient, we recorded the number, class, specificity, and MFI of all DSAs. HLA typing of donors and recipients was performed using DNA typing (Innolipa HLA Typing Kit; Innogenetics, Belgium). DSA assessment was available for 229/244 (94%) patients, HLA typing of donors being not available in 15 patients. Among 68 patients with ABMR, 60 were shown to have anti-HLA DSAs. The remaining 8 patients did not have available results for circulating anti-HLA DSAs and should be categorized as suspicious for ABMR based on Banff classification.

For the analysis, the class (I or II) and the MFI of the iDSAs were recorded. The MFI was divided into 3 categories: <1000, 1000-3000, and >3000.

Statistical Methods:

The results are presented as the means±SD for continuous variables. Frequencies of categorical variables are presented as numbers and percentages. The distribution of each protein biomarker exhibited considerable positive skewness, which was substantially reduced by use of a natural logarithm transformation.

For evaluation of the correlation of the different Banff elementary lesions and urinary biomarkers, Spearman's rank correlation coefficient (rₛ) was used, and unsupervised ascendant hierarchical clustering analysis was applied to the data set to enable visualization of the overall pattern of histological lesions and urinary biomarkers without any a priori sample classification.

We compared the levels of urinary protein biomarkers across the different diagnostic categories (i.e., ABMR, TCMR, and DNR) using the Kruskal-Wallis test followed by Dunn's post-test.

Multivariate linear regression was used to identify the Banff scores that were independently associated with urinary biomarker levels. The variables tested were i, t, v, g, ptc, cg, mm, ci, ct, cv, and ah. In a stepwise bidirectional elimination analysis, the best model according to the Bayesian Information Criterion (BIC) was retained.

ROC curves were used to illustrate the diagnostic performance of urinary biomarkers and classifier models. The discrimination ability of urinary biomarkers and the incremental value of urinary biomarkers to conventional models were evaluated by C statistics.

The association of clinical, biological, and immunological variables with the diagnosis of ABMR was evaluated by univariate and multivariate logistic regression analysis. The identified factors (i.e., all covariates associated at the P<0.1 level in the univariate analysis) were included in a final multivariate model employing stepwise backward elimination. The additive predictive value of CXCL10: Cr in combination with the reference risk model was evaluated using C statistics.

This analysis was internally validated using a 10-fold cross-validation bootstrap method. The original sample was randomly partitioned into 10 equally sized subsamples. Of the 10 subsamples, a single subsample was retained as the validation set used to test the model, and the remaining 9 subsamples were used as the training set. The cross-validation process was then repeated 10 times, with each of the 10 subsamples used exactly once as the validation data. The ten results obtained from each cross-validation were averaged to produce a single estimation.

The discrimination ability and incremental value of CXCL10: Cr were evaluated by C statistics. This analysis was repeated 1000 times using bootstrap samples to derive 95% CIs for the difference in the C statistic between models.

Cox proportional hazard analysis was used to associate the CXCL10: Cr ratio with death-censored graft survival. A Cox proportional hazards model was used to quantify the HRs and 95% CIs for the factors associated with post-biopsy kidney graft loss. Within each group of factors (i.e., clinical, biological, immunological, histological, protein biomarkers), we performed univariate and multivariate analyses with backward variable selection (parameters with P<0.20 entered into the multivariate analysis). The selected factors were entered into a single multivariate Cox model to identify the most predictive independent factors for kidney graft loss. The Kaplan-Meier method was used to estimate the cumulative incidence of graft loss, with a time scale of years since study entry (i.e., time since initial biopsy showing antibody-mediated rejection). In the survival analyses, graft survival was censored at 3 years after the index biopsy, at recipient death, or at the last visit until April 2014.

Analyses were performed with R software (version 3.1.0) and GraphPad Prism (version 5.00; GraphPad Software, San Diego, Calif.).

Results Patient and Biopsy Characteristics:

From February 2011 to January 2013, 290 matched clinically indicated biopsies and urine samples were collected from 247 kidney transplant recipients in our center. These biopsies were indicated for acute renal dysfunction (n=240), proteinuria (n=25), identification of de novo DSAs (n=13), or positive BK virus viremia (n=12).

After the exclusion of 9 biopsies from patients with BK virus nephropathy, which is known to alter urinary inflammatory signatures and for which a noninvasive biomarker would not obviate a biopsy in the presence of BK viremia, our final study sample included 281 matched biopsy/urine samples from 244 patients. Sixty seven patients had known DSAs before transplantation.

At the time of the biopsy, which was performed at a median time of 10.6 months post-transplant (interquartile range [IQR]=1.3-55.2), the serum creatinine level was 213±125 μmol/L. Seventy-eight biopsies (27.8%) revealed AR (68 ABMRs [24.2%] including 37 pure ABMRs and 31 mixed rejections, and 10 TCMRs [3.6%]). The other biopsies, subsequently classified as dysfunction with no rejection (DNR), revealed acute tubular necrosis/minimal lesions (n=43 [15%]), isolated interstitial fibrosis/tubular atrophy (n=140 [50%]), borderline lesions (n=17 [6%]), or a primary diagnosis of recurrent disease (n=3 [1%]).

Association of Acute Banff Elementary Lesions with the Urinary Biomarkers:

We assessed the level of CXCL9 and CXC10 proteins in urine samples and studied the association of CXCL9 and CXCL10 levels and values normalized to urine creatinine (CXCL9: Cr and CXCL10: Cr) with the histological findings in the 281 biopsies.

Unsupervised hierarchical clustering analysis of Banff scores and protein biomarkers showed that both CXCL9 and CXCL10 levels, normalized (left panels) or not (right panels) to urine creatinine, were highly associated with all acute Banff scores. CXCL10 and CXCL10: Cr clustered closer to microcirculation inflammatory scores (g and ptc), whereas CXCL9 and CXCL9: Cr strongly clustered with tubulointerstitial inflammatory scores (i and t).

Spearman's correlation confirmed that acute Banff scores (g, ptc, i and t) were strongly associated with urinary CXCL9, CXC9L: Cr, CXCL10, and CXCL10: Cr (all P<0.01), with Spearman's correlation coefficients, rₛ, between 0.20 and 0.41. Conversely, there was no correlation between the 4 protein biomarkers and the chronic Banff scores. Urinary CXCL9 and CXCL10 levels, normalized or not to urine creatinine, correlated well with tubulointerstitial inflammation burden (i+t score, all P<0.001). In addition, urinary CXCL9 and CXCL10 levels, normalized or not to urine creatinine, correlated well with microvascular inflammation burden (g+ptc score, all P<0.001) even after exclusion of patients with tubulointerstitial inflammation.

Because acute Banff elementary lesions were all closely correlated, multivariate linear regression was used to quantify the strength of the relationship between the urinary biomarkers and the Banff elementary lesions and to identify independent Banff scores associated with the urinary biomarkers. Multivariate regression analysis revealed that both tubulointerstitial inflammation (i and/or t scores) and microvascular inflammation (ptc score) were significantly and independently associated with urinary biomarker levels. The values of the regression coefficient β suggested that CXCL9 and the CXCL9: Cr ratio were mainly associated with tubulointerstitial inflammation, whereas CXCL10 and the CXCL10: Cr ratio were mainly associated with peritubular capillaritis. Glomerulitis did not appear as an independent factor associated with the urinary biomarkers.

Urinary Biomarkers are Diagnostic of Both TCMR and ABMR:

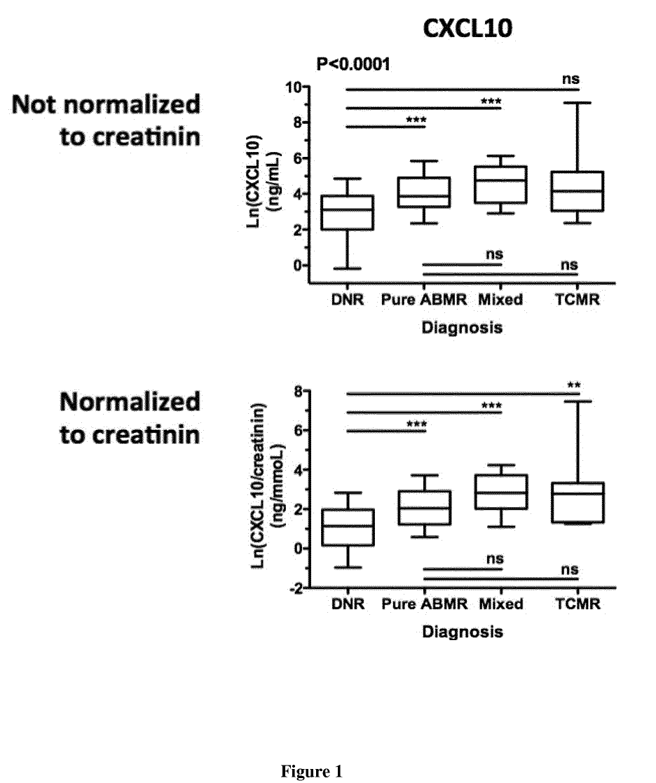

As illustrated in FIG. 1, urinary levels of the four protein biomarkers were significantly different between the diagnostic groups (all P<0.001, Kruskal-Wallis test). Compared with DNR, CXCL10 and the CXCL10: Cr ratio significantly increased in pure ABMRs (both P<0.001, Dunn's post-test) and mixed rejections (both P<0.001, Dunn's post-test). CXCL10 and the CXCL10: Cr ratio were similar in all rejection groups; however, CXCL9 and the CXCL9: Cr ratio were increased in mixed rejections (both P<0.001, Dunn's post-test) and TCMRs (both P<0.001, Dunn's post-test) but not in pure ABMRs (FIG. 1). We repeated the analysis while distinguishing borderline changes from other DNR diagnoses and highly consistent results were found with similar biomarker levels in the DNR and the Borderline groups.

Receiver operating characteristic (ROC) curve analysis was performed for each biomarker to evaluate its performance in the diagnosis of AR, pure ABMR, mixed rejection, and TCMR compared with the diagnosis of DNR. The diagnostic performance of CXCL9, CXCL9: Cr, CXCL10, and CXCL10: Cr in predicting any type of AR was similar. CXCL9 (area under the curve [AUC]=0.86; 95% CI: 0.74-0.98; P=1.2E-06) and CXCL9: Cr (AUC=0.90; 95% CI: 0.84-0.97; P=7.7E-06) were strong predictors of TCMR and that CXCL9 and CXCL9: Cr were numerically better than CXCL10 and CXCL10: Cr in the noninvasive diagnosis of TCMR. Mixed rejection was also strongly predicted by urinary chemokine levels with CXCL10 (AUC=0.80; 95% CI: 0.71-0.89; P=3.7E-08) and CXCL10: Cr (AUC=0.82; 95% CI: 0.74-0.90; P=35.5E-09) yielding the best AUC values. Importantly, urinary chemokine levels also associated with the diagnosis of pure ABMR, and again CXCL10 (AUC=0.70; 95% CI: 0.61-0.79; P=4.6E-05) and CXCL10: Cr (AUC=0.70; 95% CI: 0.61-0.79; P=4.8E-05) yielded the best AUC values.

C statistics and bootstrap validation showed that CXCL10 and the CXCL10: Cr ratio were strongly associated with ABMR, with modest positive predictive values and high negative predictive values (>91% for pure ABMR and >95% for mixed rejection), suggestive of a very low false-negative rate.

In a sensitivity analysis, we assessed the robustness of our study results by investigating the diagnostic accuracy of urine biomarkers separately in the subgroup of non-sensitized patients (e.g., patients with no identified preformed DSA) and in a subgroup analysis that included only the first biopsy of each patient, and highly consistent results were found. In another subgroup analysis, ROC curve analysis was performed to evaluate the performance of the CXCL10: Cr ratio in the diagnosis of pure ABMR compared with the DNR diagnosis. This analysis showed that the CXCL10: Cr ratio remains diagnostic of ABMR even in the total absence of associated tubulointerstitial inflammation (AUC=0.70; 95% CI: 0.61-0.79; P=4.8E-5).

Evaluation of the chemokine levels, with and without normalization to urine creatinine, revealed that the CXCL10: Cr ratio was the best marker of ABMR, and the results for this biomarker will be reported in the following sections. Potential confounding factors were assessed, and leukocyturia was identified as significant.

Urinary CXCL10: Cr Independently Improves Non-Invasive Diagnosis of ABMR:

The association of clinical, biological, immunological, and histological factors with the risk of ABMR was evaluated by univariate and multivariate logistic regression analysis. Univariate analysis showed that recipient age, use of standard criteria donor kidneys, cold ischemia time, donor age, transplantation rank, time post-transplantation, proteinuria, Ln(CXCL9), Ln(CXCL9: Cr), Ln(CXCL10), Ln(CXCL10: Cr), DSAs at the time of biopsy, and mean fluorescence intensity (MFI) of the immunodominant DSA (iDSA) were associated (P<0.1) with ABMR.

Multivariate logistic regression analysis showed that the MFI of the iDSA at biopsy (odds ratio [OR]=2.2 for MFI<1000, 95% CI: 0.8-6.0, P=0.13; OR=3.6 for MFI 1000-3000, 95% CI: 1.1-10.7, P=2.4E-2; OR=10.4 for MFI>3000, 95% CI: 4.7-24.0, P=1.3E-8) and Ln(CXCL10: Cr) (OR=1.9, 95% CI: 1.5-2.5, P=5.5E-6) were independently associated with the diagnosis of ABMR.

A 10-fold cross validation strategy was used to internally validate the model used to associate the MFI of iDSA with Ln(CXCL10: Cr). The predicted probability for each patient from the cross validation was used to construct a ROC curve. The cross-validated estimate of the AUC was 0.82 (95% CI: 0.81-0.83; p<2.2E-16). This estimate is the expected value of the AUC in an independent sample.

The inclusion of the CXCL10: Cr ratio in the reference model that used only the iDSA was found to significantly improve the noninvasive diagnosis of ABMR, with the C statistic increasing from 0.75 to 0.83 (P=0.002) with a bootstrap mean difference of 0.075 (95% CI: 0.075-0.076; P<2.2E-16). Similarly, the inclusion of the CXCL10: Cr ratio in the reference model adequately reclassified patients at lower (no event) or higher (event) risk of ABMR, which was shown by a continuous net reclassification index of 0.6718 (95% CI: 0.4004-0.9432; P<0.01). The addition of the CXCL10: Cr ratio reclassified 74 of 194 patients (38%) in the right direction in the no event group, whereas it reclassified 18 of 62 patients (29%) in the event group. The integrated discrimination improvement was 0.0854 (95% CI: 0.0488-0.122; P<0.01).

CXCL10 expression is not specific for ABMR and also increases in other types of inflammation; therefore, we repeated the analysis and addressed the improvement of prediction of any type of AR by adding urinary CXCL10 expression to the DSA measurement. The MFI of the iDSA at biopsy still predicted AR (AUC=0.72; 95% CI: 0.65-0.79; P=1.8E-10), and the inclusion of the CXCL10: Cr ratio in the model that used only the iDSA was found to significantly improve the noninvasive diagnosis of AR, with the C statistic increasing from 0.72 to 0.82 (P=0.0002) (bootstrap mean difference=0.0985; 95% CI: 0.098-0.099; P<2.2E-16).

CXCL10: Cr ratio is associated with death-censored graft loss after ABMR: Among the 68 patients with ABMR, 14 lost their graft after a median follow-up of 6 months (IQR=2-19). Death-censored graft survival after the diagnosis of ABMR was 87%, 77%, and 75% at 1, 2, and 3 years, respectively. Univariate Cox analysis of the conventional features of graft loss showed that the recipient age, donor age, serum creatinine, proteinuria, and v, i, and IF/TA scores were associated (P<0.1) with graft loss. Urinary CXCL9: Cr and CXCL10: Cr ratios were also associated with graft failure. Univariate analysis revealed that increased urinary CXCL10: Cr ratios, divided into quartiles at the time of the biopsy, correlated with graft survival in a dose-dependent manner (hazard ratio [HR]=1, 3.2, 6.0, and 10.0 for Q1, Q2, Q3, and Q4, respectively).

A multivariate Cox model identified two factors that were independently associated with graft loss, namely, proteinuria (HR=1.7; 95% CI: 1.3 to 2.2; P=2.8E-5) and CXCL10: Cr (HR=2.2; 95% CI: 1.2 to 4.0; P=8.3E-3). A Kaplan-Meier analysis of post-ABMR, death-censored graft survival showed that increased urinary CXCL10: Cr ratios at the time of biopsy correlated with graft survival.

Discussion:

Urinary biomarkers, including mRNA and protein biomarkers, have been extensively evaluated as noninvasive biomarkers of TCMR of kidney allografts. A similar strategy, however, is missing with regard to the noninvasive diagnosis of ABMR, which is currently the main cause of late allograft loss. The main observations of this study were that urinary CXCL10 levels correlated with ongoing ABMR in a large cohort of highly sensitized and well phenotyped kidney transplant recipients; combining the urinary CXCL10: Cr ratio with iDSA levels significantly improved the noninvasive diagnosis of ABMR in kidney transplant patients; and the CXCL10: Cr ratio at the time of biopsy stratified patients who were at risk of graft loss.

The chemokines CXCL9 and CXCL10 have been extensively associated with T-cell infiltrate burden, particularly tubulitis, and our demonstration that they have an increased urinary level during ABMR may be surprising. One simplistic explanation for this result could be that our ABMRs have a significant T-cell infiltrate burden, therefore contributing to an IFN-γ signature. Although low-grade interstitial infiltrate is often present in biopsies showing ABMR, this does not constitute the main explanation for the above-mentioned result, as demonstrated by restricting the analysis to ABMR cases with complete absence of interstitial infiltrate and tubulitis (i+t=0, FIG. 1). In addition, when studying the molecular signature of biopsy samples demonstrating ABMR, Sellares et al showed a clear IFN-γ signature, with CXCL10 being one of the top ABMR classifier genes. In a rat model of acute antibody-mediated endothelial injury, among several chemokines and chemokines receptors, CXCL10 was by far the most upregulated chemokine (119-fold), a result that also supports our findings.

Our multivariate linear regression analysis of the association of Banff scores with urinary biomarker levels revealed that, among microvascular inflammatory lesions, the peritubular capillaritis score, but not the glomerulitis score, appeared independently and significantly associated with the urinary chemokine level. This result suggests the prominent role of peritubular capillaritis in triggering increased level of urinary chemokines, as already suggested by Panzer et al, who found a huge up-regulation of CXCL10 in the peritubular capillaries but not in the glomerular endothelial cells in their rat model of acute antibody-mediated endothelial injury.

The accuracy of CXCL9 and CXCL10 protein in the diagnosis of ABMR has never been thoroughly evaluated. For example, in the extensive analysis conducted by the Winnipeg group, the urine CXCL10 level was associated with tubulitis, a feature of TCMR. In addition, in the recent CTOT-1 study that validated urinary CXCL9 as a diagnostic biomarker of AR, only 6 biopsies showed evidence of ABMR. Urinary CXCL9 and CXCL10 levels have also been shown to be diagnostic of BK virus-associated nephropathy.

Our analysis shows that urinary CXCL9 level is increased in TCMR and mixed rejection, confirming its close association with tubulo-interstitial inflammation. If urinary CXCL10 level is increased in TCMR and mixed rejection, we also describe for the first time its association with microvascular inflammation, mainly peritubular capillaritis, even in the total absence of tubulo-interstitial inflammation, a result that support the view that increased CXCL10 urinary levels may relate to any type of allo-immune injury. Therefore, acute allograft dysfunction with a concomitant high level of urinary CXCL10 is not synonymous with TCMR, and a biopsy is required to assess the mechanism of injury and to define adequate therapeutic interventions. This relative lack of specificity, in addition to the relatively low positive predictive values and high negative predictive values, indicates that noninvasive biomarkers may be more useful for avoiding biopsies in patients with low levels of urinary biomarkers in whom AR is highly unlikely than replace a biopsy in patients in whom AR would be anticipated based on a positive urinary marker.

Currently, the main available biomarker of ABMR is the presence of anti-HLA DSAs in the serum, and the majority of ABMRs are associated with these circulating antibodies. However, DSAs are only a risk factor for ABMR, and many patients bear DSAs without evidence of antibody-mediated injury. For instance, in our cohort, 56 out of 244 patients (23%) had DSA at time of biopsy without any histological features of ABMR. As suggested by the recent consensus guidelines on the testing and clinical management issues associated with anti-HLA antibodies in transplantation, the identification of DSAs should prompt an allograft biopsy. In this respect, the implementation of markers with high negative predictive values has the potential to avoid a large number of biopsies. Our results suggest that a single urinary chemokine, evaluated by a simple enzyme-linked immunosorbent assay (ELISA) technique, conveys a negative predictive value (NPV) of greater than 90%. Whether or not this strategy may avoid biopsies in DSA-positive renal transplant recipients will need to be evaluated prospectively.

Our study also highlights how innovative biomarkers may be implemented in the clinic to improve prognostic models that use more conventional markers. We used recommended methodological approaches for the performance and reclassification analyses²⁰ to evaluate the benefit of the urinary biomarkers when added to the conventional features, and we found that the inclusion of urinary CXCL10: Cr ratios in a conventional assessment (i.e. DSA monitoring) of kidney transplant patients improved the noninvasive diagnosis of ABMR. Molecular markers have the potential to aid in refining the diagnosis and prognosis of renal allograft outcome. Loupy et al recently demonstrated that adding the molecular microscope strategy to a conventional analysis of biopsy samples from patients with ABMR significantly improved prognostication with regard to the risk of subsequent allograft loss.¹⁶ Interestingly, CXCL10 mRNA was one of the top ABMR classifier genes. Our results suggest that urine measurement of CXCL10 may constitute a noninvasive surrogate of the ABMR molecular score and that the urinary CXCL10 protein level outperforms histological lesions with regard to the prediction of graft outcome. We believe the strategy of adding innovative biomarkers, including urinary protein markers, to those already available clinically (i.e., clinical phenotyping, DSA monitoring, and BK virus PCR analysis) would also establish robust strategies for noninvasive diagnostics and prognostics.

One third of patients included in this study had identified pretransplant DSAs, thus explaining the high rate of ABMR, which may question the generalizability of the study results. Sensitivity and specificity are known to be fixed properties of a diagnostic test, while prevalence affects positive and negative predictive values. The high rate of ABMR in our cohort was therefore the only way to demonstrate the lack of specificity of urinary chemokines in diagnosing TCMR. In addition, disease prevalence affects predictive values in that the rarer the abnormality, the greater assurance that a negative test indicates no abnormality. This finding suggests that when applied to a more conventional cohort of kidney transplant recipients, the NPV of urinary chemokines in predicting ABMR would probably be greater than 90%. This point should stimulate the evaluation of these noninvasive biomarkers in a more standard population.

The CXCL10: Cr ratio is associated with an ABMR diagnosis, although the AUC of the ROC curve of 0.75 for the diagnosis of ABMR, including pure ABMR and mixed rejection, is modest, making this assay less than ideal as a diagnostic test if considered alone and at a single time point. However, combined with the DSA level, AUC significantly improved to 0.83 and adequately reclassified patients at lower (no event) or higher (event) risk of ABMR, which was shown by a significant continuous net reclassification index and integrated discrimination improvement. Additional studies with serial urine monitoring in independent cohorts of kidney transplant recipients with more conventional immunological risk are required to assess the clinical application of this biomarker in a real life setting. In addition, our reported NPV of CXCL10: Cr of 91% for the diagnosis of pure ABMR and 96% for the diagnosis of mixed rejection is based on a single measurement of the urinary biomarker. Repeated measures and changes in the biomarker profile over time may improve the prediction of allograft status, an assumption that needs to be evaluated prospectively.

Overall, in addition to confirming their potential for the noninvasive diagnosis of tubulointerstitial inflammation, our results show, for the first time, that urinary CXCL10 levels are significantly associated with ABMR. In view of its high NPV, the CXCL10: Cr ratio will need to be evaluated in independent cohorts to assess its ability to avoid biopsies in DSA-positive patients with low levels of urinary biomarkers in whom ABMR is highly unlikely. In addition to its diagnostic potential, the CXCL10: Cr ratio, measured at the time of a biopsy showing ABMR, also identifies patients at high risk for kidney allograft loss. Overall, our results indicate that urinary CXCL10 protein is a valuable, noninvasive diagnostic and prognostic marker in kidney transplant recipients with ABMR and provides insight beyond that provided by the classical risk stratification approach that is based on DSAs and biopsy.

### EXAMPLE 2: EARLY URINARY CXCL10 IS HIGHLY PREDICTIVE OF SUBSEQUENT ACUTE REJECTION IN CLINICALLY AND HISTOLOGICALLY STABLE KIDNEY RECIPIENTS

Material & Methods

Population:

All consecutive patients who received a kidney transplant in our center from January 2010 to June 2012 were considered for this prospective, longitudinal, single-center cohort study. Patients with HIV and/or HCV infection were excluded. Other exclusion criteria consisted of primary non-function or early graft loss, death within the first 6 months, early loss of follow-up or inclusion in another protocol. The study was approved by the ethics committee of Ile-de-France XI (#13016), and all participating patients provided written informed consent.

Biopsies:

Protocol biopsies were performed at months 3 and 12 post-transplantation, and indication biopsies were performed for clinical indications (e.g., acute renal dysfunction, proteinuria, BK viremia, and de novo DSA). Biopsy specimens were fixed in formalin, acetic acid, and alcohol and were embedded in paraffin. Tissue sections were stained with hematoxylin and eosin, Masson trichrome, periodic acid Schiff reagent, and Jones reagent for light microscopy evaluation. C4d immunohistochemical staining was systematically performed (rabbit anti-human monoclonal anti-C4d, Clinisciences, 1/200 dilution).

Renal allograft biopsies were classified using the Banff 2007 update of the Banff 1997 classification.

Urine Samples:

Post-transplantation, urine was collected on day 10 and at months 1, 3, 6, 9 and 12, as well as at the time of clinically indicated renal allograft biopsies. Urine samples were centrifuged at 1,000 g for 10 minutes within 4 hours of collection. The supernatant was collected after centrifugation and stored with protease inhibitors at −80° C.

Urine Protein Analyses:

Frozen aliquots of urine supernatants were used without any dilution and tested by ELISA for CXCL10 (IP10 Quantikine ELISA, #DIP100, R&D Systems) according to the manufacturer's instructions and for CXCL9 (Human CXCL9/MIG DuoSet, R&D Systems), as recommended by the CTOT-01 study.⁸ The mean minimum detectable level for CXCL10 in the ELISA assay was 1.67 pg/mL, and urine samples with a chemokine concentration below this value were included in the analysis as half of the detection limit. For CXCL9, the urine samples with chemokine levels below the detection limit were included as half of the minimum value detected (7 pg/mL).

Measurement of urine creatinine was performed in the same sample, using a Hitachi 917 analyzer (Roche Diagnostics). The results were normalized to the urinary creatinine levels and the CXCL10:creatinine (CXCL10:Cr) and CXCL9:creatinine ratios (CXCL9:Cr) were expressed in nanograms of protein per millimole of urine creatinine.

Immunosuppression:

All patients, except for 14, received induction therapy with either rabbit ATG (Thymoglobuline®, Sanofi, France, [n=163]) or Basiliximab (Simulect®, Novartis Pharma AG, Basel, Switzerland, [n=123]). Maintenance immunosuppression consisted of a three-drug regimen including steroids (n=300) and mycophenolate mofetil (n=300) together with a calcineurin inhibitor (n=297) or everolimus (n=3). Patients with pre-existing DSA received additional intravenous immunoglobulins and, in the case of high-level DSA, prophylactic rituximab therapy and plasmapheresis.

Statistical Methods:

The results are presented as the means±SD for continuous variables. Frequencies of categorical variables are presented as numbers and percentages. The distribution of each protein biomarker exhibited considerable positive skewness, which was substantially reduced by use of a natural logarithmic transformation.

To assess the diagnostic value of urinary chemokines during clinical and subclinical acute rejection, we compared the levels of urinary protein biomarkers across the different diagnostic categories using the Mann-Whitney test, and conventional ROC curves were used to illustrate the diagnostic performance of the urinary biomarkers. The discrimination ability of urinary chemokines was evaluated by C statistics. This analysis was repeated 1000 times using bootstrap samples to derive 95% CIs.

We compared the prospective trajectories of the urinary chemokine levels in the two groups. For patients who underwent a biopsy showing AR, we included all urine samples collected from the time of transplantation until the first biopsy showing AR or until 400 days post-transplantation, whichever came first. For patients who never had a biopsy finding that showed rejection, we included all urine samples collected during the first 400 days post-transplantation.

To assess the predictive properties of urinary chemokines for subsequent AR, an endpoint that can change during follow-up, we used time-dependent ROC curve analyses.¹⁷ Time-dependent ROC curve analyses were therefore used to address the question of how well the urinary chemokine levels, assessed at 10 days, 1 month and 3 months post-transplantation, could identify subjects who developed AR before 400 days post-transplantation. To analyze the predictive value of early urinary chemokines to predict subsequent AR, patients who developed AR prior to urine sampling were excluded. The discrimination ability of CXCL9 and CXCL10:Cr were evaluated by repeating the analysis 1000 times using bootstrap samples to derive 95% CIs. We estimated the best discriminating threshold values of the urinary chemokines by maximizing the sensitivity and the specificity. In this context, the sensitivity represents the proportion of at-risk patients among those who developed AR before this date. The specificity is the proportion of risk-free patients among those who did not develop AR. Univariate and multivariate Cox proportional-hazards analysis, Kaplan-Meier analysis, and log-rank tests were used to examine the association between urinary chemokine levels and time to AR.

Analyses were performed with R software (version 3.1.0) and GraphPad Prism (version 5.00; GraphPad Software, San Diego, Calif.). Time-dependent ROC curve analyses were performed with the package ‘SurvivalROC’ on the R platform.

Results

Patient and Biopsy Characteristics:

Among the 405 patients who received a kidney allograft at Necker (Paris, France) during the inclusion period, 300 patients were prospectively included in the study. A total of 773 biopsies were performed during the first year. Protocol biopsies included 247 3-month protocol biopsies performed at a median time of 92 days post-transplant (interquartile range [IQR]=89-94 days) and 232 12-month protocol biopsies performed at a median time of 370 days post-transplant (IQR=360-385 days). A total of 294 additional indication biopsies were obtained from 185 patients at a median time of 45 days post-transplant (IQR=17-162). Overall, only 35 biopsies (4.5%) were considered of insufficient quality to yield a diagnosis. A total of 110 of the 736 informative biopsies (14.9%) revealed AR (84 pure ABMRs [11.4%], 10 mixed rejections [1.4%], and 16 TCMRs [2.2%]). Fifty-one out of the 110 AR cases were subclinical AR, diagnosed at 3 months (n=21) or 12 months (n=30).

A total of 1,619 urine samples were collected at the predetermined time points, corresponding to a mean number (±SEM) of 5.3±0.1 urine samples per patient during the first year post-transplantation. A total of 147 urine specimens, including 100 additional samples collected at the time of an indication biopsy and 47 samples collected at the time of a protocol biopsy but with concomitant acute graft dysfunction, were also included in part of the following analyses.

Diagnostic Value of Urinary Chemokines During Clinical and Subclinical Acute Rejection:

A total of 147 urine samples were collected at the time of an indication biopsy, and 434 urine samples were collected at the time of an informative protocol biopsy. These paired urine and biopsy samples allowed us to assess the diagnostic value of urinary CXCL9:Cr and CXCL10:Cr for clinical and subclinical AR. Urinary levels of CXCL9:Cr and CXCL10:Cr, measured at the time of an indication biopsy, were significantly increased in patients with clinical AR compared to patients without AR (2.40±1.94 versus 0.97±1.29, P=0.0016, Kruskal-Wallis test, and 2.00±1.22 versus 1.10±1.57, P=0.0003, respectively). Similarly, urinary levels of CXCL9:Cr and CXCL10:Cr, measured at the time of a protocol biopsy, were significantly increased in patients with subclinical AR compared to patients without AR (0.80±1.22 versus 0.47±1.16, P=0.0477, and 1.04±1.71 versus 0.27±1.82, P=0.0037, respectively). Receiver operating characteristic (ROC) curve analysis was performed for each biomarker to evaluate its performance in the diagnosis of clinical and subclinical AR, and the analysis confirmed that urinary levels of CXCL9:Cr and CXCL10:Cr are diagnostic of both clinical and subclinical AR.

Trajectories of the Chemokine Biomarkers:

To investigate the kinetics of urinary chemokine levels post-transplantation, we examined the evolution of urine CXCL9:Cr and CXCL10:Cr levels over time in the study population. We studied the loess-smoothed average prospective trajectories (i.e., the trajectories of the urine chemokines as a function of the time since transplantation) of urine CXCL9:Cr and CXCL10:Cr levels in patients who did or did not experience at least one AR episode up to 400 days post-transplantation. The trajectories for the group of patients with no diagnosed AR were flat throughout the first 400 days post-transplantation. However, progressive and early increases in the CXCL9:Cr and CXCL10:Cr levels were observed in patients who developed AR. Strikingly, urinary CXCL10:Cr levels measured during the first months post-transplantation seemed to differentiate between patients who did or did not subsequently develop AR.

Early Predictive Value of Urinary Chemokines for Subsequent Acute Rejection:

Our observation that urinary chemokine levels differed early post-transplantation in patients who developed AR prompted us to investigate whether urinary chemokine levels quantified 10 days, 1 month and 3 months post-transplantation differentiate individuals at risk of subsequent AR up to 400 days post-transplantation.

At 10 days and 1 month post-transplantation, urinary CXCL9:Cr levels remained similar in the two groups. At 3 months, urinary CXCL9:Cr levels were increased in patients with subsequent AR (1.24±1.62 vs 0.49±1.12, P=0.0298). Urinary CXCL10:Cr levels both at 1 month (1.64±1.43 versus 0.68±1.91 ng/mmoL, P=0.0005) and 3 months (1.62±1.95 versus 0.33±1.81 ng/mmoL, P=0.0009) identified patients at higher risk of developing subsequent AR.

Next, we evaluated the performance of these biomarkers for predicting subsequent AR with time-dependent ROC analyses, which were used to identify the optimal cut-off value of CXCL9:Cr and CXCL10:Cr for AR prediction at each time point. While the AUCs of the time-dependent ROC curves remained low at 10 days, the 1-month and 3-month post-transplant values for CXCL10:Cr identified patients at risk of subsequent AR, with time-dependent AUCs of 0.71 (95% CI: 0.60-0.78, P<0.001) at 1 month and 0.69 (95% CI: 0.56-0.82, P=0.003) at 3 months post-transplantation.

As early as 1 month post-transplantation, the urinary level of CXCL10:Cr predicted subsequent AR up to 400 days post-transplantation with a sensitivity of 81.6%, a specificity of 50.8%, a positive predictive value (PPV) of 29.0% and a negative predictive value (NPV) of 91.8%. We showed that the CXCL10:Cr level predicted subsequent AR with modest PPVs but high NPVs, suggestive of a very low false-negative rate.

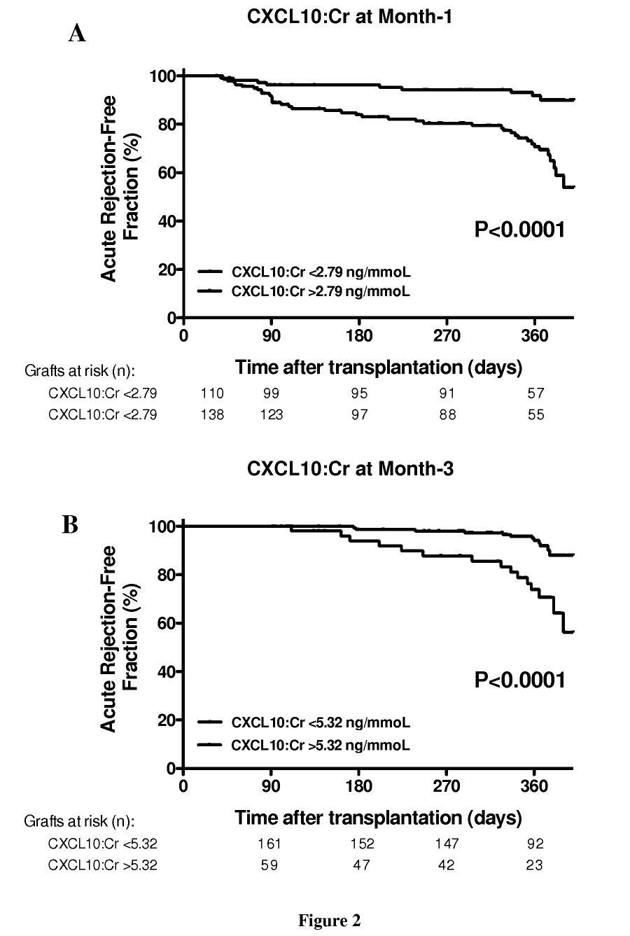

The Kaplan-Meier curves for AR-free allograft survival, stratified according to the levels of CXCL9:Cr and CXCL10:Cr that had been identified as the best cut-off points from the time-dependent ROC analyses, are shown in FIG. 2. While the optimal cut-off levels of CXCL9:Cr were ineffective for predicting subsequent AR, the cut-off levels of CXCL10:Cr at 1 month and 3 months post-transplantation strongly discriminated between patients who were at risk of AR and those who were not (both P<0.0001, log-rank test) (FIG. 2A and FIG. 2B). Strikingly, 400 days post-transplantation, the AR-free allograft survival rates were 90% and 54% in patients with urinary CXCL10:Cr levels <2.79 ng/mmoL and >2.79 ng/mmoL at 1 month, respectively (P<0.0001), and 88% and 56% in patients with urinary CXCL10:Cr levels <5.32 ng/mmoL and >5.32 ng/mmoL at 3 months, respectively (P<0.0001).

Because variable cut-off levels over time may be impractical in clinical care, we evaluated the predicted value of an intermediate cut-off point of 3.7 ng/mmoL. Using this cut-off point, the CXCL10:Cr level remained highly predictive of subsequent AR; the AR-free allograft survival rates at 400 days post-transplantation were 81% and 57% in patients with urinary CXCL10:Cr levels <3.7 ng/mmoL and >3.7 ng/mmoL at 1 month, respectively (P=0.0014), and 88% and 61%, respectively, in patients with urinary CXCL10:Cr levels <3.7 ng/mmoL and >3.7 ng/mmoL at 3 months (P=0.0004).

We then assessed the association between serially quantified chemokine levels and subsequent AR using Cox proportional hazards analysis. Univariate Cox analyses, adjusted for DSA status and history of delayed graft function (DGF), suggested that urinary CXCL9:Cr and CXCL10:Cr levels were associated with time to AR. The urinary CXCL10:Cr level strongly predicted subsequent AR at 1 month (P=0.0035) and at 3 months (P=0.0028) post-transplantation. A weaker association between the urinary CXCL9:Cr level and time to AR was observed, which reached statistical significance at 3 months post-transplantation.

Sensitivity Analyses:

Using the 3-month protocol biopsies, we tested whether urinary chemokine levels identify individuals at risk for subsequent AR independent of renal allograft histology. From the entire study population, we identified 220 kidney transplant recipients who did not develop AR during the first 3 months, who had a 3-month protocol biopsy showing no rejection, and who have available urinary CXCL10 levels. Twenty-seven of these 220 patients who were clinically and histologically stable during the first 3 months subsequently developed AR between 3 and 12 months post-transplant, with a median time of 336 days post-transplant (IQR=231-362 days); 16 of these 27 AR were subclinical AR diagnosed at one year. Patients who developed AR after the first 3 months post-transplantation were indistinguishable in their clinical, biological and histological variables at 3 months. In this subgroup analysis, univariate Cox analyses, adjusted for DSA status and history of DGF, confirmed that urinary CXCL9:Cr and CXCL10:Cr levels at 3 months post-transplantation were associated with time to AR. Multivariate Cox proportional hazard analysis, adjusted for DSA status and history of DGF, confirmed that urinary CXCL10:Cr levels, and, to a lesser extent, CXCL9:Cr levels, at 3 months post-transplantation were associated with subsequent AR occurrence, independent of concomitant histological findings.

Second, we evaluated whether the association between early urinary CXCL10 levels and subsequent AR was maintained by restricting the analysis to the prediction of subclinical AR. CXCL10:Cr values obtained 1 month post-transplantation predicted subsequent clinical AR (P=0.0157) and subclinical AR (P=0.0002) diagnosed at 3 months and 12 months post-transplantation. Similarly, at 3 months post-transplantation, urinary CXCL10:Cr levels predicted clinical AR (P=0.0004) and subsequent subclinical AR (P=0.0089) diagnosed at 12 months post-transplantation. We showed that CXCL10:Cr levels predicted subsequent clinical AR with very high NPVs (96.2% at 1 month and 97.4% at 3 months). Similarly, subclinical AR was also predicted by CXCL10:Cr levels, with NPVs of 95.3% at 1 month and 94.9% at 3 months.

Discussion:

Urinary levels of chemokines have been extensively evaluated as noninvasive biomarkers in kidney recipients. Most studies have demonstrated their ability to noninvasively diagnose concomitant clinical acute TCMR.^{4-6, 9, 12} Several studies have also suggested that they may have the ability to diagnose subclinical rejection,¹⁰⁻¹² yet very few studies have suggested that AR may be noninvasively predicted a few days or weeks before overt allograft dysfunction develops and a clinical diagnosis can be made.^{7, 8, 16} The main observations of this study were that urinary CXCL10:Cr, quantified as early as 30 days post-transplantation, is highly correlated with the subsequent risk of AR within the first 400 days post-transplantation; this predictive value is robust at 1 month and 3 months post-transplantation for predicting clinical as well as subclinical AR. Furthermore, urinary CXCL10:Cr levels assessed at 3 months, at the time of a protocol biopsy, predict subsequent AR independent of biopsy results.

Identifying noninvasive diagnostic techniques for allograft pathology is an important goal for improving patient care; predicting subsequent events is even more critical and may permit preventive therapeutic interventions to be performed. Specifically, accurate quantification of the subsequent risk of AR may help to individualize the immunosuppressive regimen in apparently stable patients. Two multicenter studies, CTOT-01 and CTOT-04,^{8, 16} provided interesting data suggesting the benefit of such an approach, showing an increase of the investigated biomarkers in the weeks before clinical AR developed. Hricik et al reported elevated urinary CXCL9 concentrations in kidney recipients with histologically diagnosed AR up to 30 days prior to clinical recognition of graft dysfunction.⁸ Suthanthiran et al demonstrated a progressive increase in the identified urinary RNA signature of patients during the 20-day period leading up to the first specimen showing acute rejection.¹⁶ Finally, Matz et al reported that urinary CXCL10 levels were predictive of clinical acute TCMR 6-7 days before a biopsy-proven diagnosis.⁷ These studies suggested that elevated urinary biomarkers predate the onset of the graft dysfunction that precipitates the for-cause biopsy. In these studies, a retrospective strategy was applied to evaluate whether the diagnostic signature existed in the previous sample collected immediately before the AR diagnosis. Our results extend these findings by demonstrating in a prospective manner that urinary CXCL10:Cr levels, quantified at 1 month and 3 months, are associated with subsequent clinical and subclinical rejection, diagnosed up to 400 days post-transplantation. Moreover, our Kaplan-Meier analyses revealed that increased levels of CXCL10:Cr were associated with rejection several months after urinary chemokine level assessment (FIG. 2). To demonstrate this association, in addition to the traditional Kaplan-Meier method and Cox univariate and multivariate models, we used a new methodological approach known as the time-dependent ROC curve, which made it possible to assess the predictive capacity of a surrogate marker.¹⁸ This approach allowed us to demonstrate that urinary chemokine data predict subsequent AR in apparently stable patients.

The early identification of kidney recipients who are at increased risk of developing AR would allow individualized adjustment of immunosuppressive regimens and exclude such patients from drug-weaning strategies. There have been many attempts to individualize immunosuppressive regimens at a few months post-transplant (steroid or calcineurin inhibitor withdrawal or switching to mTOR inhibitors); however, these studies consistently reported an increased incidence of AR after treatment modification,^{19, 20} even after a reassuring normal protocol biopsy.²⁰ We believe that our results identify an innovative approach for individual risk stratification before immunosuppressive regimen adjustments. Importantly, the predictive value of the CXCL10:Cr ratio was independent of DSA status and protocol biopsy results. In addition, as illustrated by our Kaplan-Meier analyses, the high negative predictive value of CXCL10:Cr indicates that this strategy may be used for identifying patients with a very low risk of subsequent AR. Our identified cutoff values of early urinary CXCL10:Cr levels were associated with a difference of 30-40% in the rate of AR at 400 days post-transplantation and identified patients who were at very low risk of subsequent clinical AR.

Our finding that the urinary CXCL10:Cr level at 3 months is associated with subsequent AR in clinically stable patients, independent of conventional parameters including DSA and protocol biopsy, reinforces the notion that classical risk assessment can be improved by innovative noninvasive strategies. Strikingly, we show that the urinary CXCL10:Cr level at 3 months is an independent predictor of subsequent AR risk, reflecting the presence of a “sub-histological” disease state with a significant predictive value for future AR. This hypothesis is strengthened by previous molecular studies. Naesens et al²¹ examined the transcriptome of 24 kidney allografts in stable patients with no AR at 6 months, 12 of whom demonstrated worsened chronic damage, evaluated by the CADI score (Chronic Allograft Damage Index) and 12 remained histologically stable at 2 years. Immune-related genes were significantly overexpressed in the 6-month biopsies of the progressor group. Our results suggest that urine measurements of CXCL10:Cr may be a noninvasive surrogate for the sub-histological inflammatory burden and that the urinary CXCL10:Cr level outperforms histological lesions for predicting AR.

Consistent with this finding, this study shows that urinary CXCL10:Cr predicts subsequent AR in patients who often demonstrate antibody-mediated changes. Importantly, our results suggest that the CXCL10:Cr level predicts subsequent clinical as well as subclinical AR, suggesting that this surrogate marker has a high sensitivity.

Finally, even if no firm mechanistic insights arise from this clinical association study, it is surprising to see that patients who will develop AR during the first post-transplant year have a clear urinary signal as soon as 30 days post-transplantation, suggesting that minimal inflammatory burden is actually present very soon after transplantation in patients who will experience rejection. At day 10 post-transplantation, non-adjusted Cox models suggested an association between urinary chemokine levels and time to AR, a result that was no longer observed after adjustment for a history of DGF, thus reflecting that chemokine levels very soon post-transplant may be altered by ischemia-reperfusion injury.

Overall, in addition to its previously reported diagnostic value as a non-invasive urinary biomarker of TCMR and ABMR, our results show, for the first time, that urinary CXCL10 levels, quantified early post-transplantation, predict the subsequent risk of AR within the first 400 days following transplantation, independent of protocol biopsy results. These results support the view that longitudinal monitoring of urine chemokine biomarkers may improve risk stratification and allow for greater treatment individualization.

### REFERENCES

Throughout this application, various references describe the state of the art to which this invention pertains. The disclosures of these references are hereby incorporated by reference into the present disclosure.
1. G. Kidney Disease: Improving Global Outcomes Transplant Work: KDIGO clinical practice guideline for the care of kidney transplant recipients. American journal of transplantation: official journal of the American Society of Transplantation and the American Society of Transplant Surgeons 9 Suppl 3: S1-155, 2009
2. D. Anglicheau, M. Suthanthiran: Noninvasive prediction of organ graft rejection and outcome using gene expression patterns. Transplantation 86: 192-199, 2008
3. U. Panzer, O. M. Steinmetz, R. R. Reinking, T. N. Meyer, S. Fehr, A. Schneider, G. Zahner, G. Wolf, U. Helmchen, P. Schaerli, R. A. Stahl, F. Thaiss: Compartment-specific expression and function of the chemokine IP-10/CXCL10 in a model of renal endothelial microvascular injury. Journal of the American Society of Nephrology: JASN 17: 454-464, 2006
4. H. Hu, J. Kwun, B. D. Aizenstein,S. J. Knechtle: Noninvasive detection of acute and chronic injuries in human renal transplant by elevation of multiple cytokines/chemokines in urine. Transplantation 87: 1814-1820, 2009
5. H. Hu, B. D. Aizenstein, A. Puchalski, J. A. Burmania, M. M. Hamawy, S. J. Knechtle: Elevation of CXCR3-binding chemokines in urine indicates acute renal-allograft dysfunction. American journal of transplantation: official journal of the American Society of Transplantation and the American Society of Transplant Surgeons 4: 432-437, 2004
6. I. A. Hauser, S. Spiegler, E. Kiss, S. Gauer, O. Sichler, E. H. Scheuermann, H. Ackermann, J. M. Pfeilschifter, H. Geiger, H. J. Grone,H. H. Radeke: Prediction of acute renal allograft rejection by urinary monokine induced by IFN-gamma (MIG). Journal of the American Society of Nephrology: JASN 16: 1849-1858, 2005
7. M. Matz, J. Beyer, D. Wunsch, M. F. Mashreghi, M. Seiler, J. Pratschke, N. Babel, H. D. Volk, P. Reinke, K. Kotsch: Early post-transplant urinary IP-10 expression after kidney transplantation is predictive of short- and long-term graft function. Kidney international 69: 1683-1690, 2006
8. D. E. Hricik, P. Nickerson, R. N. Formica, E. D. Poggio, D. Rush, K. A. Newell, J. Goebel, I. W. Gibson, R. L. Fairchild, M. Riggs, K. Spain, D. Ikle, N. D. Bridges, P. S. Heeger, C. consortium: Multicenter validation of urinary CXCL9 as a risk-stratifying biomarker for kidney transplant injury. American journal of transplantation: official journal of the American Society of Transplantation and the American Society of Transplant Surgeons 13: 2634-2644, 2013
9. J. J. A: Urinary Chemokines CXCL9 and CXCL10 Are Noninvasive Markers of Renal Allograft Rejection and BK Viral Infection. American journal of transplantation: official journal of the American Society of Transplantation and the American Society of Transplant Surgeons 2228-2234, 2011
10. S. Schaub, P. Nickerson, D. Rush, M. Mayr, C. Hess, M. Golian, W. Stefura, K. Hayglass: Urinary CXCL9 and CXCL10 levels correlate with the extent of subclinical tubulitis. American journal of transplantation: official journal of the American Society of Transplantation and the American Society of Transplant Surgeons 9: 1347-1353, 2009
11. J. Ho, D. N. Rush, M. Karpinski, L. Storsley, I. W. Gibson, J. Bestland, A. Gao, W. Stefura, K. T. HayGlass, P. W. Nickerson: Validation of urinary CXCL10 as a marker of borderline, subclinical, and clinical tubulitis. Transplantation 92: 878-882, 2011
12. P. Hirt-Minkowski, P. Amico, J. Ho, A. Gao, J. Bestland, H. Hopfer, J. Steiger, M. Dickenmann, F. Burkhalter, D. Rush, P. Nickerson,S. Schaub: Detection of clinical and subclinical tubulo-interstitial inflammation by the urinary CXCL10 chemokine in a real-life setting. American journal of transplantation: official journal of the American Society of Transplantation and the American Society of Transplant Surgeons 12: 1811-1823, 2012
13. T. D. Blydt-Hansen, I. W. Gibson, A. Gao, B. Dufault, J. Ho: Elevated Urinary CXCL10-to-Creatinine Ratio Is Associated With Subclinical and Clinical Rejection in Pediatric Renal Transplantation. Transplantation 2014
15. H. J.: Validation of Urinary CXCL10 As a Marker of Borderline, Subclinical, and Clinical Tubulitis. Transplantation 82: 878-882, 2011
16. M. Suthanthiran, J. E. Schwartz, R. Ding, M. Abecassis, D. Dadhania, B. Samstein, S. J. Knechtle, J. Friedewald, Y. T. Becker, V. K. Sharma, N. M. Williams, C. S. Chang, C. Hoang, T. Muthukumar, P. August, K. S. Keslar, R. L. Fairchild, D. E. Hricik, P. S. Heeger, L. Han, J. Liu, M. Riggs, D. N. Ikle, N. D. Bridges, A. Shaked, I. Clinical Trials in Organ Transplantation 04 Study: Urinary-cell mRNA profile and acute cellular rejection in kidney allografts. The New England journal of medicine 369: 20-31, 2013
17. L. E. Chambless, G. Diao: Estimation of time-dependent area under the ROC curve for long-term risk prediction. Statistics in medicine 25: 3474-3486, 2006
18. P. J. Heagerty, T. Lumley, M. S. Pepe: Time-dependent ROC curves for censored survival data and a diagnostic marker. Biometrics 56: 337-344, 2000
19. K. Budde, T. Becker, W. Arns, C. Sommerer, P. Reinke, U. Eisenberger, S. Kramer, W. Fischer, H. Gschaidmeier, F. Pietruck,Z. S. Investigators: Everolimus-based, calcineurin-inhibitor-free regimen in recipients of de-novo kidney transplants: an open-label, randomised, controlled trial. Lancet 377: 837-847, 2011
20. Y. Lebranchu, A. Thierry, O. Toupance, P. F. Westeel, I. Etienne, E. Thervet, B. Moulin, T. Frouget, Y. Le Meur, D. Glotz, A. E. Heng, C. Onno, M. Buchler, S. Girardot-Seguin,B. Hurault de Ligny: Efficacy on renal function of early conversion from cyclosporine to sirolimus 3 months after renal transplantation: concept study. American journal of transplantation: official journal of the American Society of Transplantation and the American Society of Transplant Surgeons 9: 1115-1123, 2009
21. M. Naesens, P. Khatri, L. Li, T. K. Sigdel, M. J. Vitalone, R. Chen, A. J. Butte, O. Salvatierra, M. M. Sarwal: Progressive histological damage in renal allografts is associated with expression of innate and adaptive immunity genes. Kidney international 80: 1364-1376, 2011

1. A method for determining whether a renal transplant patient is at risk of acute rejection, comprising the steps of (i) determining the expression level of the CXCL10 polypeptide in a urine sample obtained from said patient up until 3-months post-transplantation, and (ii) comparing said expression level with a predetermined reference value, wherein an expression level that is lower than the predetermined reference value indicates that said patient is not at risk of acute rejection or that said patient has a decreased risk of acute rejection.

2. The method according to claim 1, wherein the expression level of the CXCL10 polypeptide is expressed as the ratio of expression levels of CXCL10 polypeptide to urinary creatinine (CXCL10:Cr).

3. The method according to claim 1, wherein the urine sample is obtained 10-days post-transplantation, 1-month post-transplantation, and/or 3-months post-transplantation.

4. A method for diagnosing antibody-mediated rejection (ABMR) in a renal transplant patient, comprising the steps of (i) determining the expression level of the CXCL10 polypeptide in a urine sample obtained from said patient, and (ii) comparing said expression level with a predetermined reference value, wherein an expression level of the CXCL10 polypeptide determined at step (i) that is lower than the predetermined reference value indicates that said patient does not have ABMR.

5. The method according to claim 4, wherein the expression level of the CXCL10 polypeptide is expressed as the ratio of expression levels of CXCL10 polypeptide to urinary creatinine (CXCL10:Cr).

6. A method for adjusting the immunosuppressive treatment administered to a renal transplanted patient following its transplantation, comprising the steps of:
(i) performing the method for determining whether a renal transplant patient is at risk of acute rejection according to claim 1,
and when the expression level is higher than the predetermined reference,
(ii) concluding that said patient has or is at risk of acute rejection, and
(iii) providing an effective amount of an immunosuppressive treatment to the patient.

7. A method for preventing antibody-mediated rejection or progression of ABMR in a renal transplanted patient, comprising the steps of: (i) performing the method for diagnosing ABMR according to claim 4, and, if said patient is found to have ABMR, then (ii) administering to said patient a therapeutically effective amount of a compound selected from the group consisting of anti-thymocyte globulin, monoclonal anti-CD20 antibodies, proteasome inhibitor and anti-C5 antibodies, and/or performing plasmapheresis.

8. The method of claim 6, wherein the immunosuppressive treatment comprises administering to the patient a therapeutically effective amount of a compound selected from the group consisting of anti-thymocyte globulin, monoclonal anti-CD20 antibodies, proteasome inhibitor and anti-C5 antibodies and/or performing plasmapheresis.


SEQUENCE LISTING (1 sequence)

SEQ ID NO: 1 - protein, 98 aa, Homo sapiens
MNQTAILICCLIFLTLSGIQGVPLSRTVRCTCISISNQPVNPRSLEKLEIIPASQFCPRVEIIATMKKKGEKRCLNPESKAIKNLLKAVSKERSKRSP